(19) United States
(12) Patent Application Publication
(10) Pub. No.: US 2024/0189241 A1
(43) Pub. Date: Jun. 13, 2024

(54) PHOSPHOLIPIDS AS ANION CHELATING AGENTS IN PHARMACEUTICAL FORMULATIONS

(71) Applicant: R.P. SCHERER TECHNOLOGIES, LLC, Carson City, NV (US)

(72) Inventors: Giang Hoang Thuy Au, Tampa, FL (US); William Derek Bush, Belleair, FL (US)

(21) Appl. No.: 18/284,916

(22) PCT Filed: Mar. 31, 2022
(86) PCT No.: PCT/US2022/022737

§ 371 Date: Sep. 29, 2023

Related U.S. Application Data
(60) Provisional application No. 63/169,330, filed on Apr. 1, 2021.

(51) Int. Cl. A61K 9/48

(52) U.S. Cl. CPC A61K 9/4858; A61K 9/4825

(57) ABSTRACT

Disclosed herein are dosage forms including a fill material that incorporates an anionic chelating agent, such as lecithin, and a salt form of a basic or acidic active pharmaceutical ingredient and a free ion. The molar ratio of the anionic chelating agent, such as lecithin, to the free ion ranging from about 0.5 to about 3. Also disclosed herein are methods of stabilizing dosage forms that include a salt form of a basic or acidic active pharmaceutical ingredient, methods of preparing such dosage forms, and methods of using such dosage forms.

The present application claims priority to U.S. Provisional Patent Application No. 63/169,330 filed on Apr. 1, 2021, the contents of which are incorporated by reference in its entirety.

### FIELD OF THE DISCLOSURE

The present disclosure relates to the field of pharmaceutical compositions that comprise a basic or acidic active pharmaceutical ingredient and a free anion. The present invention is also related to methods of preparing and methods of using such pharmaceutical compositions.

### BACKGROUND OF THE DISCLOSURE

Oral administration is one of the most preferable routes for drug administration. A sufficiently high amount of drug needs to be dissolved in the gastro-intestinal (GI) tract, absorbed and distributed via the blood stream to reach the target site. More than 40% of newly discovered chemical entities are poorly soluble (BSC class II), poorly permeable (BSC class III), or both (BCS class IV). Because of poor solubility, these drugs have low dissolution and limited absorption. Enhancing the dissolution of the drug in the GI tract is a challenge in drug discovery. A compound with low solubility, less than 100 μg/mL in aqueous solution, is usually considered dissolution-limited. Multiple techniques have been used in effort to increase the aqueous solubility of a drug. Among these techniques, salt formation of the API is a popular approach in drug discovery, particularly for basic compounds.

The specific salts of active pharmaceutical ingredients (APIs) are often formed to achieve desirable formulation properties. Depending on the functional groups present on the API, a potential counterion can be selected to create the salt form. Salt formation can be used for APIs with low melting temperature (usually being a liquid in free base form) to increase their melting temperatures and maintain the stable crystalline state. Salt formation is also well-known technique to increase aqueous solubility or lipophilicity of a drug molecule depending on the delivery vehicle and the drug's purpose (Gupta D., Bhatia D., Dave V., Sutariya V., Gupta S. V. “Salts of Therapeutic Agents: Chemical, Physicochemical and Biological Considerations.” Molecules 23; 1719-1734 (2018), hereinafter “Gupta et, al., 2018”). Salts of acidic drugs have been commonly made using sodium (Na+) as counterion, whereas chloride (from hydrochloric acid) is a common counterion for basic drugs (Vioglio P. C., Chierotti M. R., Gobetto R. “Pharmaceutical aspects of salt and cocrystal forms of APIs and characterization challenges.” Advanced Drug Delivery Reviews, 117: 86-110 (2017), hereinafter “Vioglio et al., 2017”).

Acidic or basic counterions in the salt form API can alter the pH of the microenvironment in liquid dosage forms. Reactivity between the API and excipients can be influenced by the changes in pH which can lead to degradation of the API and generate significant impurities in drug products. The changes in pH with free ions can also affect the integrity of the carrier such as gelatin shell of a drug product. The use of a chelating agent to stabilize chemicals and drugs in solution is a common strategy. The chelating agent can bind the counterions/free ions and stabilize both physical and chemical properties of the formulation.

Most commonly, ion complexation focuses on heavy metal chelating in pharmaceutical formulations with ethylenediaminetetraacetic acid (EDTA) and its salt being effective metal ion chelators. However, the most common chelators that are used in pharmaceutical applications are cation chelators. There are currently very limited options for anionic chelators for pharmaceutical drug products that have shown to be both safe and effective.

There is a need for safe and effective anionic chelators that could be used to formulate pharmaceutical composition of basic or acidic APIs with free anions while maintaining the stability of the composition over an extended duration.

### OBJECTS AND SUMMARY OF THE DISCLOSURE

It is an object of certain embodiments of the present disclosure to provide a dosage form comprising a basic APT and a free anion, wherein the dosage form is stable over an extended duration.

It is an object of certain embodiments of the present disclosure to provide a dosage form comprising an acidic API and a free anion, wherein the dosage form is stable over an extended duration.

It is another object of certain embodiments of the present disclosure to provide a method for stabilizing a dosage form comprising a basic API and a free anion over an extended duration.

It is another object of certain embodiments of the present disclosure to provide a method for stabilizing a dosage form comprising an acidic API and a free anion over an extended duration.

It is a further object of certain embodiments of the present disclosure to provide a method for preparing a dosage form comprising a basic API and a free anion, wherein the dosage form is stable over an extended duration.

It is a further object of certain embodiments of the present disclosure to provide a method for preparing a dosage form comprising an acidic API and a free anion, wherein the dosage form is stable over an extended duration.

It is yet another object of certain embodiments of the present disclosure to prepare a dosage form that maximizes the amount of basic or acidic API in the dosage form and minimizes the amount of non-value adding excipients in the dosage form.

The above objects of the present disclosure and others may be achieved by the present disclosure which is directed to a capsule with a fill material encapsulated in a shell composition, wherein the fill material includes an anionic chelating agent comprising lecithin with a salt form of a basic or acidic API and a free anion at a molar ratio of lecithin to the free anion of about 0.5 to about 3. In certain embodiments, the free anion is at least one of: chloride, bromide, fluoride, sulfate, phosphate, formate, acetate, trifluoroacetate, maleate, tartrate, methanesulfonate, benzenesulfonate, nitrate, or a combination thereof. In certain embodiments, the lecithin includes from about 10 wt % to about 95 wt % phospholipid components with positively charged functional groups (e.g., phosphatidylcholine (PC), phosphatidylethanolamine (PE), or a mixture thereof), based on total weight of the lecithin.

In certain embodiments, the present disclosure is directed to a capsule with a fill material encapsulated in a shell composition, wherein the fill material includes an anionic chelating agent comprising lecithin having from about 10 wt % to about 95 wt % phospholipid components with positively charged functional groups (e.g., phosphatidylcholine (PC), phosphatidylethanolamine (PE), or a mixture thereof), based on total weight of the lecithin, a salt form of a basic or acidic API, and a free anion.

In certain embodiments, the capsules of the present disclosure maintain their integrity at 40° C. over three weeks, wherein the integrity of the capsule is measured based on leakage of the fill material from the shell composition.

In some embodiments, the present disclosure is directed to a method for stabilizing a capsule. In certain embodiments, the method includes combining a salt form of a basic or acidic active pharmaceutical ingredient (API) and a free anion with an anionic chelating agent comprising lecithin at a molar ratio of lecithin to free anion of about 0.5 to about 3 to prepare a fill material; and encapsulating the fill material in a shell composition. In certain embodiments, the stabilized capsule maintains its integrity at 40° C. over three weeks, wherein the integrity of the capsule is measured based on leakage of the fill material from the shell composition.

In some embodiments, the present disclosure is directed to a method for preparing a dosage form (e.g., a hard or a softgel capsule). In certain embodiments, the method may comprise preparing any of the fill materials described herein, which include a salt form of a basic or acidic API, a free anion, and an ionic chelating agent, and encapsulating the fill material in a shell composition.

In some embodiments, the present disclosure is directed to a method of treatment comprising administering to a patient in need thereof a therapeutically effective amount of any of the dosage forms disclosed herein.

### Definitions

As used herein, the singular forms “a,” “an,” and “the” include plural references unless the context clearly indicates otherwise. Thus, for example, reference to “an active pharmaceutical ingredient” includes a single active pharmaceutical ingredient as well as a mixture of two or more different active pharmaceutical ingredients, and reference to an “excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number 10%, such that “about 10” would include from 9 to 11.

As used herein, the terms “active agent,” “active ingredient,” “active pharmaceutical ingredient.” and “drug” refer to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is nonsuperimposable on its mirror image and hence optically active wherein the enantiomer rotates the plane of polarized light in one direction by a certain degree, and its mirror image rotates the plane of polarized light by the same degree but in the opposite direction.

The term “chiral center” refers to a carbon atom to which four different groups are attached.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of an active agent. The terms “treatment of” and “treating” includes the lessening of the severity of or cessation of a condition or lessening the severity of or cessation of symptoms of a condition.

The terms “prevention of” and “preventing” includes the avoidance of the onset of a condition.

“Therapeutically effective amount” is intended to include an amount of an active agent, or an amount of the combination of active agents. e.g., to treat or prevent the condition, or to treat the symptoms of the condition, in a subject.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “extended release” refers to an active agent that is released over a period of time, e.g., to provide a once daily or twice daily dosage form.

The term “immediate release” refers to a dosage form that allows the drug to dissolve in the gastrointestinal tract, with no intention of delaying or prolonging the dissolution or absorption of the drug. For instance, to the release of at least 85%, at least 90%, or at least 95% of an active agent in about 5 minutes, about 15 minutes, about 30 minutes, about 45 minutes or about 60 minutes, as measured by in-vitro dissolution in a USP Apparatus 1 (#40 mesh basket), in a USP Apparatus 2 (paddle), or in a USP Apparatus 3 (reciprocating cylinder) in aqueous media (pH 1-8) at room temperature.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 depicts the free chloride anion chelating capabilities of different levels of phosphatidylcholine (PC) in various lecithin grades.

FIG. 2A depicts a softgel capsule formulation without lecithin before an accelerated stability study and after the accelerated stability study.

FIG. 2B depicts a softgel capsule formulation with lecithin before an accelerated stability study and after the accelerated stability study.

### DETAILED DESCRIPTION

### Dosage Forms

According to various embodiments, the present disclosure is related to a dosage form including a fill material encapsulated in a shell composition. In certain embodiments, the dosage form may be a capsule, such as, without limitations, a hard capsule or a soft capsule (e.g., a softgel capsule).

In certain embodiments, the fill material includes a salt form of a basic or acidic active pharmaceutical ingredient (API) and a free anion. In certain embodiments, the free anion is chloride, bromide, fluoride, sulfate, phosphate, formate, acetate, trifluoroacetate, maleate, tartrate, methanesulfonate, benzenesulfonate, nitrate, or a combination thereof. The term “salt form” of the basic or acidic API refers to a compound that contains a negatively charged counterion (anion), such as chloride (e.g., in hydrochloric acid). In certain embodiments, the free anion may be derived from a pharmaceutically acceptable salt that may include, but not be limited to, inorganic acid salts such as hydrochloride, hydrobromide, sulfate, phosphate and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like. In certain embodiments, the free anion may be derived from pharmaceutically acceptable salts that include organic amine salts such as triethylamine salt, pyridine salt, picoline salt, ethanolamine salt, triethanolamine salt, dicyclohexylamine salt, N,N′-dibenzylethylenediamine salt and the like.

In one embodiment, the salt form of the basic or acidic API is the HCl salt form of the API and the free anion is chloride. In one embodiment, the salt form of the basic or acidic API is HBr salt form of the API and the free anion is bromide. In one embodiment, the salt form of the basic or acidic API is the HF salt form of the API and the free anion is fluoride. In one embodiment, the salt form of the basic or acidic API is sulfuric acid (H₂SO₄) form of the API and the free anion is hydrogen sulfate (HSO₄_{^{−}}) or sulfate (SO₄_{^{2−}}). In one embodiment, the salt form of the basic or acidic APT is the phosphoric acid salt form of the APT and the free anion is a phosphate. In one embodiment, the salt form of the basic or acidic API is the formic acid salt form of the API and the free anion is formate. In one embodiment, the salt form of the basic or acidic API is the acetic acid salt form of the API and the free anion is acetate. In one embodiment, the salt form of the basic or acidic API is the trifluoroacetic acid salt form of the API and the free anion is trifluoroacetate. In one embodiment, the salt form of the basic or acidic API is the maleic acid salt form of the API and the free anion is maleate. In one embodiment, the salt form of the basic or acidic API is the tartaric acid salt form of the API and the free anion is tartrate. In one embodiment, the salt form of the basic or acidic API is the methanesulfonic acid salt form of the API and the free anion is methanesulfonate. In one embodiment, the salt form of the basic or acidic API is the benzenesulfonic acid salt form of the API and the free anion is benzenesulfonate. In one embodiment, the salt form of the basic or acidic API is the nitric acid salt form of the API and the free anion is nitrate.

The basic or acidic API may be present in the fill material at a concentration ranging from 0.0001 w/w % to 90.0 w/w %, for examples, from about 0.001 w/w %, about 0.01 w/w %, about 0.1 w/wo, about 0.5 w/w %, about 1.0 w/w %, about 3.0 w/w %, about 5.0 w/w %, about 8.0 w/w %, or about 10.0 w/w % to about 15.0 w/w %, about 20.0 w/w %, about 25.0 w/w %, about 30.0 w/w %, about 35.0 w/w %, about 40.0 w/w %, about 50.0 w/w %, about 60.0 w/w %, about 70.0 w/w %, about 80.0 w/w %, or about 90.0 w/w %, based on total weight of the fill material. In certain embodiments, the dosage forms described herein enable inclusion of a greater concentration of the basic or acidic API relative to comparative dosage forms that do not include an anion chelating agent in the fill material. This may be so because comparative dosage forms that do not include an anion chelating agent in the fill material may not be chemically or physically stable due to the presence of a high concentration of the free anion arising from the salt form of the basic or acidic API. In contrast, the anionic chelating agent in the fill material of the dosage forms described herein may reduce the concentration of the free anion arising from the salt form of the basic or acidic API by complexing with it. This may enable inclusion of more basic or acidic API in the fill material while still maintaining the chemical and physical stability of the dosage form over time.

Dosage forms with a higher concentration of the API than the concentration that is currently available on the market may be beneficial as it may contribute to a decrease in the number of dosage form units a patient take or to a decrease in the number of times a patient takes the dosage form (for instance, if a dosage form that is currently on the market comprises about 4 mg of the API for a twice daily administration, a dosage form according to the present disclosure may comprise about 8 mg of the API for a once a day administration).

Suitable APIs include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, and combinations thereof.

In certain embodiments, suitable APIs include, without limitations, ibuprofen, diclofenac, dextromethorphan, choline, combinations thereof and the like.

In certain embodiments, the fill material further includes an anionic chelating agent. Certain phospholipids (e.g., phosphatidylcholine) may be candidates as anionic salt form (such as HCl salt) APT chelators. All lipids that contain phosphorus are called phospholipids. Phospholipids are surface-active, amphiphilic molecules, which comprise of a polar head group and a lipophilic tail. The phospholipid molecule structure includes a glycerol backbone, which is esterified in positions 1 and 2 with fatty acids and in position 3 with phosphate. Exemplary phospholipids include phosphatidylcholine (PC), phosphatidylethanolamine (PE), phosphatidylinositol (PI), and phosphatidylserine (PS). PC and PE are zwitterionic and have a neutral charge at pH of 7.

A mixture of phosphatides, such as, PC, PE, PS, and PI, combined with various amounts of other substances such as triglycerides, fatty acids, and carbohydrates, as separated from the crude vegetable oil source, and containing not less than 50% of acetone-insoluble matter, is defined as Lecithin per United States Pharmacopoeia (USP) definition (Hoogevest P, and Wendel A. “The use of natural and synthetic phospholipids as pharmaceutical excipients.” European Journal of Lipid Science and Technology, 116(9): 1088-1107 (2014), hereinafter “van Hoogevest et, al, 2014”).

In certain embodiments, the phospholipid components in lecithin, such as phosphatidylcholine (PC) and phosphatidylethanolamine (PE), can act as anionic chelating agent. The positive charge on the tertiary and quaternary amine can bind to the free anion produced by salt form of the basic or acidic APT (such as chloride anions) and minimize the impact of the free anion on the chemical stability of the fill material and of the shell composition (that encapsulates the fill material).

In certain embodiments, the fill material includes the free anion (from the salt form of the basic or acidic API) and lecithin (or other source of anionic chelating agent) at an amount that provides for a molar ratio of the anionic chelating agent to the free anion of about 0.5 to about 3. In certain embodiments, the molar ratio of the anionic chelating agent to the free anion ranges from any of about 0.5, about 0.8, about 1.0, about 1.2, about 1.5, or about 1.8 to any of about 2.0, about 2.2, about 2.4, about 2.6, or about 2.8, or any range or value in therein.

In certain embodiments, the fill material includes the API and phosphatidylcholine (or other source of anionic chelating agent) at an amount that provides for a molar ratio of the anionic chelating agent (e.g., phosphatidylcholine) to the API of about 1:1 to about 1:50, about 1:2 to about 1:45, about 1:3 to about 1:40, about 1:4 to about 1:35, about 1:5 to about 1:30, or about 1:5 to about 1:25, or any sub-range or single value therein. In certain embodiments, the lecithin includes from about 10 wt % to about 95 wt % phospholipid components with a positively charged functional group (e.g., phosphatidylcholine (PC) and phosphatidylethanolamine (PE)), based on total weight of the lecithin. In one embodiment, the lecithin includes from about 10 wt % to about 95 wt % phosphatides comprising one or more of phosphatidylcholine (PC), phosphatidylethanolamine (PE), phosphatidylinositol (PI), and phosphatidylserine (PS), based on total weight of the lecithin.

In one embodiment, the lecithin includes phosphatidylcholine (PC), phosphatidylethanolamine (PE), or a mixture thereof (e.g., about 10 wt % to about 95 wt %, based on total weight of the lecithin). In one embodiment, the lecithin includes phosphatidylcholine (PC) (e.g., about 10 wt % to about 95 wt %, based on total weight of the lecithin).

In certain embodiments, the concentration of phospholipid components, or phospholipid components with a positively charged functional group, or of any one or more of PC, PE, PS, or PI may range from any of about 10 wt %, about 15 wt %, about 20 wt %, about 25 wt %, about 30 wt %, about 35 wt %, about 40 wt %, or about 45 wt % to any of about 50 wt %, about 55 wt %, about 60 wt %, about 65 wt %, about 70 wt %, about 75 wt/O, about 80 wt/O, about 85 wt %, or about 90 wt %, or any range or value therein, based on total weight of the lecithin.

In certain embodiments, the concentration of the anionic chelating agent in the fill material may range from any of about 1 wt %, about 3 wt %, about 5 wt %, about 8 wt %, about 10 wt %, about 12 wt %, about 15 wt %, about 18 wt %, about 20 wt %, or about 25 wt % to any of about 28 wt %, about 30 wt %, about 33 wt %, about 35 wt %, about 38 wt %, about 40 wt %, about 45 wt %, or about 50 wt %, or any value or range therein, based on the total weight of the fill material.

It is believed, without construing this theory is limiting, that the anionic chelating agent binds to the free anion produced by the salt form of the basic or acidic API, which in turn minimizes the free anion's impact on the chemical stability of the fill material and the physical stability of the shell composition. Hence, in certain embodiments, the dosage forms described herein are chemically and physically stable over an extended duration. The dosage form may be stored at elevated temperatures and/or elevated humidity and may still maintain chemical and physical stability over time. In certain embodiments, the dosage forms described herein (e.g., capsules described herein) maintain their integrity at 40° C., and three weeks of storage. The term “integrity” with reference to a capsule is measured based on the leakage of the fill material from the shell composition of the capsule. A capsule maintains its integrity when there is no leakage of the fill material from the shell composition.

In certain embodiments, a shell composition encapsulates the fill material. The fill material may be in a liquid or in a semi-solid form and the shell composition may be used to administer the fill material. In one embodiment, the capsule is a softgel capsule and the shell composition includes gelatin, such as, without limitations, Type A gelatin (derived from an acid hydrolysis process), Type B gelatin (derived from an alkaline hydrolysis process), or a combination thereof. In certain embodiments, the capsule is a hard capsule and the shell composition includes carrageenan.

In addition to gelatin and/or carrageenan, the shell composition may further include at least one of a plasticizer, water, starch, colorant, or a combination thereof. For instance, in one embodiment, the dosage form is a soft capsule with a shell composition including gelatin, plasticizer, water, and colorant(s). In another embodiment, the dosage form is a hard capsule with a shell composition including carrageenan, plasticizer, starch, water, and colorant(s).

The dosage form may be in a form suitable for administration via an oral route, sublingual route, buccal route, vaginal route, or rectal route. In some embodiments, the final dosage form may have a shape selected from, without limitations, the group consisting of round, oval, oblong, capsule, tube, and teardrop. In some embodiments, the final dosage form has a single compartment. In other embodiments, the final dosage form has multiple compartments (also referred to as chambers). For instance, the final dosage form may have two, three, four, or more chambers.

In certain embodiments, the fill material may include further excipients and/or fillers. For instance, the fill material may further include lipid-based matrix comprising one or more of glycerides, triglycerides, semi-synthetic ester glycerides, fatty acids, alcohols, fatty acid esters, lipophilic surfactants, hydrophilic surfactants, carbohydrates, and co-solvents, and combinations thereof.

In certain embodiments, the fill material includes a lipid-based matrix that includes vegetable oils. The vegetable oils may be present in the fill material at a concentration ranging from any of about 10 wt/o, about 15 wt %, about 20 wt %, about 25 wt %, about 30 wt %, about 35 wt %, about 40 wt %, or about 45 wt % to any of about 50 wt %, about 55 wt %, about 60 wt %, about 65 wt %, about 70 wt %, about 75 wt %, about 80 wt %, about 85 wt %, or about 90 wt %, or any range or value therein, based on total weight of the fill material. In certain embodiments, the vegetable oils may include from any of about 55 wt %, about 60 wt %, about 65 wt %, about 70 wt %, or about 75 wt % to any of about 80 wt %, about 85 wt %, about 90 wt %, or about 95 wt % glycerides of polyunsaturated fatty acids, based on total weight of the vegetable oils. Exemplary suitable vegetable oils include, without limitations, one or more of olive oil, sesame oil, corn oil, peanut oil, safflower oil, soybean oil, or a combination thereof.

In certain embodiments, the fill material includes a lipid-based matrix that includes fatty acids with the formulation R—C(═O)—OH, wherein R is C₄-C₂₀, and wherein the fatty acids are fully saturated or contain one or more sites of unsaturation. The fatty acids may be present in the fill material at a concentration ranging from any of about 10 wt %, about 15 wt %, about 20 wt %, about 25 wt %, about 30 wt %, about 35 wt %, about 40 wt %, or about 45 wt % to any of about 50 wt %, about 55 wt %, about 60 wt %, about 65 wt %, about 70 wt %, about 75 wt %, about 80 wt %, about 85 wt %, or about 90 wt %, or any range or value therein, based on total weight of the fill material. Exemplary suitable fatty acids include, without limitations, one or more of oleic acid, linoleic acid, myristic acid, stearic acid, lauric acid, palmitic acid, or a combination thereof.

In certain embodiments, the fill material includes a lipid-based matrix that includes one or more of alcohols, fatty acid esters, or a combination thereof.

The alcohols may be present in the fill material at a concentration ranging from any of about 0.5 wt %, about 1 wt %, about 5 wt %, about 10 wt %, about 15 wt %, about 20 wt %, about 25 wt %, about 30 wt %, about 35 wt/o, about 40 wt %, or about 45 wt % to any of about 50 wt %, about 55 wt %, about 60 wt %, about 65 wt %, about 70 wt %, about 75 wt %, about 80 wt %, about 85 wt %, or about 90 wt %, or any range or value therein, based on total weight of the fill material. Exemplary suitable alcohols include, without limitations, one or more of ethanol, isopropanol, isobutanol, glycerol, propylene glycol, or a combination thereof.

Exemplary suitable fatty acid esters include, without limitations, one or more of mono-, di-, tri-esters of medium chain or long-chain fatty acid surfactants and/or co-solvents, or a combination thereof. Exemplary suitable surfactants and/or co-solvents include, without limitations, one or more of polyoxyl 35 castor oil, polyoxyl 40 hydrogenated castor oil, polyethylene glycol, polysorbate 80, span 80, labrafil M2125, labrasol, gelucire 44/14, or a combination thereof.

Suitable surfactants with an HLB value of less than 10 may be selected, without limitations, from the group consisting of ethylene oxide/propylene oxide (EO/PO) copolymers, glycerol monocaprylate, glycerol monocaprate, glycerol caprylate/caprate, glycerol monooleate, glycerol monostearate, glycerol laurate, glycerol monolinoleate, glycerol behenate, glycerol palmitostearate, petroleum and lanolin alcohols, polyoxyethylene alkyl ethers (e.g., polyoxyl 4 lauryl ether, polyoxyl 2 cetyl ether, polyoxyl 2 stearyl ether, polyoxyl 2 oleyl ether), sorbitan fatty acid esters (e.g., sorbitan monoisostearate, sorbitan monolaurate, sorbitan monopalmitate, sorbitan monostearate, sorbitan sesquistearate, sorbitan sesquioleate, sorbitan sesquistearate, sorbitan diisostearate, sorbitan dioleate, sorbitan triisostearate, sorbitan trioleate, sorbitan tristearate), sucrose esters, poly(ethylene glycol)-block-poly(propylene glycol)-block-poly(ethylene glycol) (pluronic copolymers), lechitin, phospholipids, steareth-2, oleth-2, ceteth-2, PEG-30 dipolyhydroxystearate, propylene glycol monocaprylate, propylene glycol dilaurate, propylene glycol monolaurate, propylene glycol monostearate, propylene glycol isostearate, alpha tocopherol, mixed tocopherols, tricaprylin, nonionic emulsifying waxes, anionic emulsifying waxs, sorbitan monooleate, sorbitan monostearate, sorbitan monopalmitate, sorbitan tristearate, sorbitan trioleate, and combinations thereof.

Exemplary suitable plasticizers that may be used in the fill material and/or in the shell composition include, without limitations, alcohol plasticizer such as isomalt, maltitol, sorbitol, xylitol, erythritol, adonitol, dulcitol, pentaerythritol, or mannitol; or polyol plasticizer such as glycerin, diglycerin, ethylene glycol, diethylene glycol, triethyleneglycol, tetraethylene glycol, dipropylene glycol, a polyethylene glycol up to 10.000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

Colorants may also be referred to herein as “dye or pigment” or as a “coloring agent.” Colorants refer to a substance that imparts coloring and/or aesthetic appearance to the dosage form. A dye is a colored substance that has an affinity to the substrate to which it is being applied. The dye may be applied in an aqueous solution, and requires a mordant to improve the fastness of the dye on the substrate. A pigment is a material that changes the color of reflected or transmitted light as the result of wavelength-selective absorption. This physical process differs from fluorescence, phosphorescence, and other forms of luminescence, in which a material emits light. Both dyes and pigments appear to be colored because they absorb some wavelengths of light more than others. In contrast with a dye, a pigment generally is insoluble, and has no affinity for the substrate.

Exemplary colorants that may be in the dosage form may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

In certain embodiments, the dosage forms described herein may include additional pharmaceutically acceptable fillers and/or excipients, such as, without limitations, fats with high melting point (e.g., triglycerides with a melting point greater than 25° C.), waxes, oils with low melting point (e.g., triglycerides with a melting point below 25° C.), liquid lipids, surfactants with HLB values greater than 10, solvents, cosolvents, solid high molecular weight polyethylene glycol, liquid polyethylene glycol, lubricants, pore formers, dispersing agents, gelatin, gums, water-soluble polymers, water, glycerin, sorbitol, cyclodextrins, flavoring agents, disintegrants, and combinations thereof. In some embodiments, the dosage form may comprise additional excipients such as solubility enhancers, solubilizers (e.g., caprylocaproyl polyoxyl-8 glycerides, and polyethylene glycol monostearate), bioavailability enhancers, plasticizers, colorants, opacifying agents, fragrances, enzymes, sweeteners, spices, vitamins, preservatives, stabilizers, antioxidants, release agents (e.g., lipid matrix for extended release such as glyceryl distearate), extenders, cross-linking agents, antiblocking agents, detackifying agents, diluents, antifoams, buffering agents, blowing agents, bulking agents, adjuvants, flow accelerators, mold release agents, granulating agents, binders, oils/fats, pH modifiers, absorbents, glidants (e.g., silicon dioxide), adhesives, anti-adherents (e.g, talc, cornstarch, colloidal silicone dioxide (Cab-O-Sil™), DL-Leucine, sodium lauryl sulfate, and metallic stearates), acidulants, softeners, resins, demulcents, emulsifiers, osmotic agents, elastomers, bleaching agents (e.g., sodium metabisulfite, sodium bisulfite or others), aversive agents such as bitterants (e.g., denatonium salts such as denatonium benzoate, denatonium saccharide, and denatonium chloride; sucrose octaacetate; quinine; flavonoids such as quercetin and naringen; and quassinoids such as quassin and brucine) and pungents (e.g., capsaicin, piperine, allyl isothiocyanate, and resinferatoxin), combinations thereof, and other functional ingredients, in amounts suitable for their intended purposes. Suitable excipients may be in a liquid, semi-solid, and/or solid form.

The fillers and/or excipients may independently or cumulatively be present in the dosage form at a concentration of about 50 wt/o or less, about 40 wt % or less, about 30 wt % or less, about 20 wt % or less, about 15 wt % or less, about 10 wt/o or less, about 5 wt % or less, about 4 wt/o or less, about 3 wt/o or less, about 2 wt % or less, about 1 wt % or less, about 0.5 wt % or less, about 0.1 wt % or less, based on the total weight of the dosage form. In some embodiments, the dosage form may have no fillers (e.g., 0 wt %). In some embodiments, the dosage form may have no excipients (e.g., 0 wt %). In some embodiments, the dosage form may comprise fillers and/or excipients in an amount ranging. e.g., from about 2 wt % to about 50 wt %, from about 6 wt % to about 40 wt %, from about 10, wt % to about 30 wt/o, from about 10 wt % to about 40 wt %, from about 15 wt % to about 35 wt %, from about 20 wt % to about 30 w %, from about 20 wt % to about 25 wt %, or from about 15 wt % to about 25 wt %, individually or collectively, based on the total weight of the dosage form.

### Flavoring Agents

“Flavoring agent” refers to a substance capable of providing a flavor. In addition to providing a palatable and pleasurable factor to the user, the flavoring agent can also mask undesirable flavors present in the dosage form. The flavoring agent can include natural flavoring agents (e.g., extracts).

“Flavor extract” refers to a flavoring agent obtained by extracting a part of a raw material, e.g., animal or plant material, often by using a solvent such as ethanol or water. The majority of natural essences are obtained by extracting the essential oil from the blossoms, fruit, roots, etc., or the whole plants, through four techniques: expression (when the oil is very plentiful and easily obtained, as in lemon peel), absorption (generally accomplished by steeping in alcohol, as vanilla beans), maceration (used to create smaller bits of the whole, as in making peppermint extract, etc.), and distillation (used with maceration, but in many cases, it requires expert chemical knowledge and the erection of costly stills).

Exemplary flavoring agents that may be in the dosage form may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

### Fragrances

Exemplary fragrances that may be in the dosage form include, but are not limited to, natural and/or synthetic fragrance raw materials. For instance, oil soluble perfume oils, which may or may not be in mixture with water soluble perfume oils. Oil soluble perfume materials are natural, or natural-identical essential oils such as orange oil, lavender oil, pine oil, eucalyptus oil, lemon oil, clove leaf, peppermint oil, cedarwood oil, rosemary oil, bergamot oil, lavandin oil, patchouli oil, chamomile oil, jasmine oil, spike oil, rose oil, Vetiver oil, fennel oil, anise oil, thyme oil, germanium oil, menthol, and marjoram oil. An animal fragrance is for example musk, castoreum, aber or zibet. Spagyric essences are also known in the art. They are made by fermenting certain herbs that are then processed to the final product. Synthetic fragrance ingredients are for example synthetic essential oils such as composed of single compounds such as linalol, terpineol, nerol, citronellal, benzaldehyde, cinnamon aldehyde, vanillin, ethylvanillin, or methylacetophenone. The fragrance materials may also be synthetic oil soluble perfume oils selected from the usual group consisting of fragrant hydrocarbons, alcohols, ketones, aldehydes, ethers, esters, polyene derivatives. Other fragrances that may be used are catalogued and described in references and databases such as S. Arctander, Perfume and Flavor Chemicals, Volumes I and II (1960, 1969; reprint 2000); Allured's Flavor and Fragrance Materials (2005); and database maintained by the Research Institute for Fragrance Materials at wwv.rifm.org.

### Sweeteners

The term “sweetener” refers to a substance capable of providing a palatable and pleasurable factor to the user, and/or capable of masking undesirable flavors present in the dosage form. Exemplary sweeteners that may be in the dosage form may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet@ and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

### Vitamins

As used herein, the term “vitamin” refers to an organic compound required by an organism as a vital nutrient in limited amounts. An organic chemical compound (or related set of compounds) is called a vitamin when it cannot be synthesized in sufficient quantities by an organism, and must be obtained from the diet. Thus, the term is conditional both on the circumstances and on the particular organism. For example, ascorbic acid (Vitamin C) is a vitamin for humans, but not for most other animals, and biotin and vitamin D are required in the human diet only in certain circumstances.

Exemplary human vitamins that may be in the dosage form may include, but not be limited to, Vitamin A (e.g., retinol, retinal, and four carotenoids including beta carotene), Vitamin B1 (thiamine), Vitamin B2 (riboflavin), Vitamin B3 (e.g., niacin and niacinamide), Vitamin B5 (pantothenic acid), Vitamin B6 (e.g., pyridoxine, pyridoxamine, and pyridoxal), Vitamin B7 (biotin), Vitamin B9 (e.g., folic acid and folinic acid), Vitamin B12 (e.g., cyanocobalamin, hydroxocobalamin, and methylcobalamin), Vitamin C (ascorbic acid), Vitamin D (cholecalciferol), Vitamin E (e.g., tocopherols and tocotrienols), and Vitamin K (e.g., phylloquinone, phytonadione, and menaquinones).

### Preservative

The term “preservative”, as used herein, refers to an agent that extends the storage life of the dosage form by retarding or preventing deterioration of flavor, odor, color, texture, appearance, therapeutic value, or safety. A preservative need not provide a lethal, irreversible action resulting in partial or complete microbial cell destruction or incapacitation. Sterilants, sanitizers, disinfectants, sporicides, viracides and tuberculocidal agents provide such an irreversible mode of action, sometimes referred to as “bactericidal” action. In contrast, a preservative can provide an inhibitory or bacteriostatic action that is reversible, in that the target microbes can resume multiplication if the preservative is removed. The principal differences between a preservative and a sanitizer primarily involve mode of action (a preservative prevents growth rather than killing microorganisms) and exposure time (a preservative has days to months to act whereas a sanitizer has at most a few minutes to act).

### Antioxidants

Exemplary antioxidants that may be in the dosage form may include, but not be limited to, sterically hindered phenols, aryl amines, thioureas, thiocarbamates, phosphites, thioether esters, and combinations of the foregoing. Other suitable examples of antioxidants include, but are not limited to, alkylated monophenols, including but not limited to, 2,6-di-tert-butyl-4-methylphenol, 2-tert-butyl-4,6-di-methylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, nonylphenols which are linear or branched in the side chains, for example, 2,6-di-nonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1-yl)phenol and mixtures thereof, alkylthiomethylphenols, including but not limited to, 2,4-dioctylthiomethyl-6-tert-hutylphenol, 2,4dioctylthiomethyl-6-methylphenol, 2,4-dioetylthiomethyl-6-ethylphenol, 2,6-di-dodecylthiomethyl-4-nonylphenol, hydroquinones and alkylated hydroquinones, including but not limited to, 2,6-di-tert-hutyl-4-methoxyphenol, 2,5-di-tert-butylhydroquinone, 2,5-di-tort-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate, tocopherols, including but not limited to, α-tocopherol, β-tocopherol, γ-tocopherol, δ-tocopherol and mixtures thereof (vitamin E), hydroxylated thiodiphenyl ethers, including but not limited to, 2,2′-thiobis(6-tort-butyl-4-methylphenol), 2,2′-thiobis(4-oetylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)-disulfide, alkylidenebisphenols, including but not limited to, 2,2′-methylenebis(6-tert-butyl-4-methylphenol), 2,2′-methylenebis(6-tert-butyl-4-ethylphenol), 2,2′-methylenebis[4-methyl-6-(α-methylcyclohexyl)-phenol], 2,2′-methylenebis(4-methyl-6-cyclohexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol), 4,4′-methylenebis(6-tert-butyl-2-methylphenol), 1,1-bis(5-tert-butyl-4-hydroxy-2-methylphenyl)butane, 2,6-bis(3-test-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy-2-methyl-phenyl)-3-n-dodecylmercaptobutane, ethylene glycol bis[3,3-bis(3′-tert-butyl-4′-hydroxyphenyl)butyrate], bis(3-tert-butyl-4-hydroxy-5-methyl-phenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis(5-tert-butyl-4-hydroxy-2-methylphenyl)-4-n-dodecylmercaptobutane, 1,5,5-tetra-(5-tert-butyl-4-hydroxy-2-methylphenyl)pentane. O-, N- and S-benzyl compounds, including but not limited to, 3,5,3′,5′-tetra-tert-butyl,-4,4′-dihydroxydibenzyl ether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxvbenzyl)sulfide, isooctyl-3,5-di-tert-butyl-4-hydroxybenzylmercaptoacetate, hydroxybenzylated malonates, including but not limited to, dioctadecyl-2,2-bis(3,5-di-tert-butyl-2-hydroxvbenzyl)malonate, di-octadecyl-2(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate, aromatic hydroxybenzyl compounds, including but not limited to, 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol, triazine compounds, including but not limited to, 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris-(3,5-di-tert-butyl-4-hydroxyphenoxy)-1,2,3-triazine, 1,3,5-tris(3,5-di-tert-butyl-4-hydroxybenzyl)isocyanurate, 1,3,5-tris(4-tert-butyl-3-hydroxy-2,6-dimethylbenzyl)isocyanurate, 2,4,6-tris-(3,5-di-tert-butyl-4-hydroxyphenylethyl)-1,3,5-triazine, 1,3,5-tris(3,5-di-tert-butyl-4-hydroxy-phenylpropionyl)-hexahydro-1,3,5-triazine, 1,3,5-tris(3,5-dicyclohexyl-4-hydroxybenzyl)iso-cyanurate, benzylphosphonates, including but not limited to, dimethyl-2,5-di-tert-butyl-4-hydroxybenzylphosphonate, diethyl-3,5-di-tert-butyl-4-hydroxybenzylphosphonate, dioctadecyl 3,5-di-tert-butyl-4-hydroxybenzylphosphonate, dioctadecyl-5-tert-butyl-4-hydroxy-3-methylbenzylphosphonate, the calcium salt of the monoethyl ester of 3,5-di-tert-butyl-4-hydroxybenzylphosphonic acid, acylaminophenols, including but not limited to, 4-hydroxylauranilide, 4-hydroxystearanilide, octyl N-(3,5-di-tert-butyl-4-hydroxyphenyl)carbamate, esters of 0-(3,5-di-tert-butyl-4-hydroxyphenyl)propionic acid with mono- or polyhydric alcohols, e.g. with methanol, ethanol n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate. N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, esters of β-(5-tert-butyl-4-hydroxy-3-methylphenyl)propionic acid with mono- or polyhydric alcohols, e.g. with methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis-(hydroxyethyl)oxamide, 3-thiaundecanol, 3-thiapentadecanol, trimethylhexanediol trimethylolpropane, 4-hydroxymethyl-1-phospha-2,6,7-trioxabicyclo[2.2.2]octane; 3,9-bis[2-{3-(3-tert-butyl-4-hydroxy-5-methylphenyl)propionyloxy}-1,1-dimethylethyl]-2,4,8,10-tetraoxaspiro(5.5]-undecane, esters of 6-(3,5-dicyclohexyl-4-hydroxyphenyl)propionic acid with mono- or polyhydric alcohols, e.g. with methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2,2,2]octane, esters of 3,5-di-tert-butyl-4-hydroxyphenyl acetic acid with mono- or polyhydric alcohols, e.g. with methanol, ethanol, octanol, octadecanol, 1,6-hexanediol, 1,9-nonanediol, ethylene glycol, 1,2-propanediol, neopentyl glycal, thiodiethyl.ene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo(2.2.2]octane, amides of 6-(3,5-di-tert-butyl-4-hydroxyphenyl)propionic acid e.g. N,N′-bis(3,5-di-tert-butylA-hydroxyphenylpropionyl)hexamethylenediamide, N,N′-bis(3,5-di-tert-butyl-4-hydroxyphenylpropionyl)trimethylenediamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard®XL-1, supplied by Uniroyal), ascorbic acid (vitamin C), aminic antioxidants, including but not limited to, N,N′-di-isopropyl-p-phenylenediamine, N,N′-di-sec-butyl-p-phenylenediamine, N,N′-bis(1,4-dimethylpentyl)-p-phenylenediamine. N,N′-bis(I-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine. N,N′-bis(2-naphthyl)-p-phenylenediamine, N-isopropyl-N′-phenyl-p-phenylenediamine. N-(1,3-dimethylbutyl)-N′-phenyl-p-phenylenediamine, N-(1-methylheptyI)—N′-phenyl-p-phenylenediamine, N-cyclohexyl-N′-phenyl-p-phenylenediamine, 4(p-toluenesulfamoyl)diphenylamine, N,N′-dimethyl-N,N′-di-sec-butyl-p-phenylenediamine, diphenylamine, N-allyldiphenylamine, 4-isopropoxydiphenylamine. N-phenyl-1-naphthylamine, N-(4-tert-octylphenyl)-1-naphthylamine, N-phenyl-2-naphthylamine, octylated diphenylamine, including but not limited to, p,p′-di-tert-octyldiphenylamine, 4-n-butylaminophenol, 4-butyrylaminophenol, 4-nonanoylaminophenol, 4-dodecanoylaminophenol, 4-octadecanoylaminophenol, bis(4-methoxyphenyl)amine 2,6-di-tert-butyl-4-dimethylaminomethylphenol, 2,4′-dianinodiphenylmethane, 4,4′-diaminodiphenylmethane, N,N,N′,N′-tetramethyl-4,4′-diaminodiphenylmethane, 1,2-bis[(2-methylphenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyldiphenylamines, a mixture of mono- and dialkylated teak-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octyl-phenothiazines, N-allylphenothiazine, N,N,N′,N′-tetraphenyl-1,4-diaminobut-2-ene, and combinations of the foregoing.

### Lubricants/Release Agents

Suitable lubricants/release agents for the dosage form can include, but are not limited to, fatty acids and their salts, fatty alcohols, fatty esters, fatty amines, fatty amine acetates and fatty amides. Other suitable lubricants may include, but not be limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and camauba wax, silica, fumed silica, colloidal silica, calcium stearate, long chain fatty alcohols, boric acid, sodium benzoate and sodium acetate, sodium chloride. DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

### Extenders/Antiblocking Agents/Detackifying Agents

Suitable extenders/antiblocking agents/detackifying agents for the dosage form can include, but are not limited to, starches, modified starches, crosslinked polyvinylpyrrolidone, crosslinked cellulose, microcrystalline cellulose, silica, metallic oxides, calcium carbonate, talc and mica.

### Diluents

Suitable diluents useful in the dosage forms according to the disclosure include, but are not limited to, lactose USP, lactose USP (anhydrous), lactose USP (spray dried), starch USP, directly compressible starch, mannitol USP, sorbitol, dextrose monohydrate, microcrystalline cellulose NF, dibasic calcium phosphate dihydrate NF, sucrose-based diluents, confectioner's sugar, monobasic calcium sulfate monohydrate, calcium sulfate dihydrate NF, calcium lactate trihydrate granular NF, dextrates NF (e.g., Emdex™), dextrose (e.g., Cerelose™), inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

### Oils/Fats

Exemplary oils and fats that may be in the dosage form may include, but not be limited to, almond oil, argan oil, avocado oil, canola oil, cashew oil, castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, walnut oil, and watermelon seed oil. Other oil and fats that may be in the fill of the PVA shell may include, but not be limited to, fish oil (omega-3), crill oil, animal or vegetable fats, e.g., in their hydrogenated form, mono-, di-, and tri-glycerides with C₁₂-, C₁₄-, C₁₆-, C₁₈-, C₂₀- and C₂₂-fatty acids.

pH modifiers

Exemplary pH modifiers that may be in the dosage form may include, but not be limited to, hydrochloric acid, potassium hydroxide, sodium hydroxide, ammonium hydroxide, sulfuric acid, phosphoric acid, and nitric acid.

### Other Excipients

Other exemplary excipients that may be in the dosage form may include, but not be limited to, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids, polyacrylanides, and polyacrylic acid esters, polymethacrylic acids, polymethacrylamides, and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers; inorganic fillers, such as the oxides of magnesium aluminum, silicon, titanium, etc.

Other pharmaceutically acceptable excipients that may be used in the dosage form may include, without limitations, a hydrophobic material, including, but is not limited to, digestible, long chain (C₈-C₅₀, especially C₁₂-C₄₀), substituted or unsubstituted hydrocarbons, such as natural or synthetic waxes (such as beeswax, glycowax, castor wax and camauba wax), fatty alcohols (such as lauryl, myristyl, stearyl, cetyl or preferably cetostearyl alcohol), fatty acids, including, but not limited to, mono-diglyceride of medium chain fatty acids (such as caprylic, capric, caproic, lauric, oleic, linoleic), medium chain triglycerides, fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones.

Additional pharmaceutically acceptable excipients may further include polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, acetic acid, caprylic acid, oleic acid, polyaminoacids or peptides, polyamides, polyacrylamide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), methacrylic acid/methyl methacrylate, methacrylic acid/ethyl acrylate copolymers, methacrylic acid/methyl acrylate/methyl methacrylate copolymers, shellac, hydroxypropyl methylcellulose phthalate, hydroxyl propyl methyl cellulose acetate succinate, hydroxypropyl methyl cellulose trimellitate, cellulose acetate phthalates, polyvinyl acetate phthalates, PEG-35 castor oil, caprylocaproyl polyoxyl-8 glycerides, glyceryl distearate, and combinations thereof.

### Release Rate

The dosage forms disclosed herein may exhibit an immediate release profile.

In certain embodiments, the dosage forms disclosed herein release at least about 80%, at least about 85%, at least about 90%, at least about 95%, or at least about 98% of the active agent within 15 minutes as measured by in-vitro dissolution in a USP Apparatus 1 (#40 mesh basket), in a USP Apparatus 2 (paddle), or in a USP Apparatus 3 (reciprocating cylinder) in aqueous media (at a pH ranging from about 1 to about 8) at about 37° C.

In certain embodiments, the dosage forms disclosed herein release at least about 80%, at least about 85%, at least about 90%, at least about 95%, or at least about 98% of the active agent within 30 minutes as measured by in-vitro dissolution in a USP Apparatus 1 (#40 mesh basket), in a USP Apparatus 2(paddle), or in a USP Apparatus 3 (reciprocating cylinder) in aqueous media (at a pH ranging from about 1 to about 8) at 37° C.

In certain embodiments, the dosage forms disclosed herein release at least about 80%, at least about 85%, at least about 90%, at least about 95%, or at least about 98% of the active agent within 45 minutes as measured by in-vitro dissolution in a USP Apparatus 1 (#40 mesh basket), in a USP Apparatus 2(paddle), or in a USP Apparatus 3 (reciprocating cylinder) in aqueous media (at a pH ranging from about 1 to about 8) at 37° C.

In certain embodiments, the dosage forms disclosed herein release at least about 80%, at least about 85%, at least about 90%, at least about 95%, or at least about 98% of the active agent within 60 minutes as measured by in-vitro dissolution in a USP Apparatus 1 (#40 mesh basket), in a USP Apparatus 2(paddle), or in a USP Apparatus 3 (reciprocating cylinder) in aqueous media (at a pH ranging from about 1 to about 8) at room temperature.

The dosage forms disclosed herein may exhibit an extended release profile.

In certain embodiments, the dosage form disclosed herein may release about 10 wt % to about 30 wt % of active agent at 1 hours, about 25 wt % to about 50 wt % of active agent at 2 hours, about 40 wt % to about 80 wt % of active agent at 4 hours, about 65 wt % to about 95 wt % of active agent at 8 hours, from about 80 wt % to about 100 wt % at 12 hours, and greater than 90 wt % of active agent at 24 hours, in each case, as measured by an in-vitro dissolution in a USP Apparatus 1 (basket) at 100 rpm, in a USP Apparatus 2 (paddle) at 50 rpm, 75 rpm, or 100 rpm, or in a USP Apparatus 3 (reciprocating cylinder) in aqueous media (at a pH 1-8) at 37° C.

In certain embodiments, the dosage form disclosed herein may release about 15 wt % to about 25 wt % of active agent at 1 hours, about 30 wt % to about 40 wt % of active agent at 2 hours, about 55 wt % to about 75 wt % of active agent at 4 hours, about 75 wt % to about 85 wt % of active agent at 8 hours, from about 90 wt % to about 100 wt % at 12 hours, and greater than 95 wt % of active agent at 24 hours, in each case, as measured by an in-vitro dissolution in a USP Apparatus 1 (basket) at 100 rpm, in a USP Apparatus 2 (paddle) at 50 rpm, 75 rpm, or 100 rpm, or in a USP Apparatus 3 (reciprocating cylinder) in aqueous media (at a pH 1-8) at 37° C.

### Methods of Preparation, Stabilization, and Treatment

In some embodiments, the present invention is directed to a method for stabilizing and/or preparing any of the dosage forms described herein. The method comprises combining a salt form of a basic or acidic API and a free anion with an anionic chelating agent. The anionic chelating agent may include lecithin or another source of a phospholipid component with a positively charged functional groups (such as PC or PE). In certain embodiments, the molar ratio of the anionic chelating agent to the free anion (that is provided by the basic or acidic API) may range from any of about 0.5, about 0.8, about 1.0, about 1.2, about 1.5, or about 1.8 to any of about 2.0, about 2.2, about 2.4, about 2.6, about 2.8, or about 3.0 or any range or value in therein. In one embodiment, the molar ratio of the anionic chelating agent to the free anion may range from about 0.5 to about 3.0.

In certain embodiments, the molar ratio of the anionic chelating agent (e.g., PC) to the API may range from any of about 1:1 to about 1:50, about 1:2 to about 1:45, about 1:3 to about 1:40, about 1:4 to about 1:35, about 1:5 to about 1:30, or about 1:5 to about 1:25, or any sub-range or value in therein.

In certain embodiments, the method for stabilizing and/or preparing any of the dosage forms described herein may further include encapsulating any of the fill materials described herein in any of the shell compositions described herein.

In certain embodiments, the methods of preparation and/or stabilization described herein contribute to the formation of dosage forms that are chemically and physically stable over an extended duration. In certain embodiments, dosage forms prepared and/or stabilized by the methods described herein may be stored at elevated temperatures (e.g., 40° C.) and/or elevated humidity (e.g., relative humidity of about 75%) and may still maintain chemical and physical stability (e.g., integrity) over time (e.g., about three weeks).

In certain embodiments, the methods of stabilization and/or preparation described herein may form a dosage form, such as a capsule, that maintains its integrity even under accelerated stability study conditions (e.g., elevated temperature and/or humidity) over an extended duration ranging from any of about 1 day, about 2 days, about 3 days, about 4 days, about 5 days, about 6 days, or about 7 days to any of about 8 days, about 9 days, about 10 days, about 11 days, about 12 days, about 13 days, about 14 days, about 15 days, about 16 days, about 17 days, about 18 days, about 19 days, about 20 days, or about 21 days.

The accelerated stability study according to embodiments herein may be performed at a temperature ranging from about 25° C., to about 40° C., and at a humidity ranging from, e.g., about 60% to about 75%. In certain embodiments, the accelerated stability study may be performed at a temperature of about 40° C., and a relative humidity of about 75%.

In some embodiments, the method for stabilizing and/or preparing any of the dosage forms described herein may further comprise dissolving or suspending a basic or acidic API in a homogenous mixture or in a matrix that includes one or more of the components of the fill material.

In certain embodiments, the method for stabilizing and/or preparing any of the dosage forms described herein may further comprise dosing the dissolved or suspended basic or acidic API in the homogenous mixture or in the matrix into preformed cavities using a rotary die machine. The dosed blister cavities may then be cooled and sealed. This approach may eliminate the need for fillers, thereby maximizing the amount of solubility and/or bioavailability enhancing materials used.

In other embodiments, the method for stabilizing and/or preparing any of the dosage forms described herein may further comprise filling the dissolved or suspended basic or acidic API within the homogenous mixture or within the matrix into a softshell capsule or into a hardshell capsule (e.g., a soft-gelatin capsule or a starch- or a carrageenan- based capsule).

In some embodiments, the present invention is directed to a method for preparing a dosage form that includes mixing a basic or acidic API, a free anion, an anionic chelating agent, and at least one solid or semisolid lipid to form a mixture. The method may further comprise heating the mixture to melt the at least one solid or semisolid lipid to form a molten mixture. The method may further comprise forming the molten mixture into a dosage form and curing the dosage form. In one embodiment, forming the molten mixture into a dosage form may comprise dosing the molten mixture into a preformed blister cavity. In one embodiment, forming the molten mixture into a dosage form may comprise encapsulating the molten mixture in a hardshell capsule or a softshell capsule.

In some embodiments, the present invention is directed to a method of treatment comprising administering to a patient in need thereof a therapeutically effective amount of any of the dosage forms disclosed herein.

### ILLUSTRATIVE EXAMPLES

The following examples are set forth to assist in understanding the disclosure and should not be construed as specifically limiting the disclosure described and claimed herein. Such variations of the disclosure, including the substitution of all equivalents now known or later developed, which would be within the purview of those skilled in the art, and changes in formulation or minor changes in experimental design, are to be considered to fall within the scope of the disclosure incorporated herein.

### Example 1—Lecithin Chelation Capability

Different levels of phosphatidylcholine (PC) in various lecithin grades were evaluated for anion chelating capability, particularly for free chloride anions chelating capabilities. Increasing the amount of PC in the formulation can potentially bind more of the free chloride ions (Cl) and minimize the migration of the chloride ions into the gelatin shell. Lecithin amount was varied using the same base formulation to evaluate the effect of increasing PC level. Three different Lecithin grades were also evaluated: Liquid Lecithin (13% PC), Lipoid S45 NF (45% PC) and Phospholipon 90G (90% PC).

An electrode probe specific for the chloride ion was used to measure the free chloride ion activity while adjusting the amount of lecithin in the formulation. As the percent of lecithin increased, the percent of free chloride ion dropped in the formulations tested.

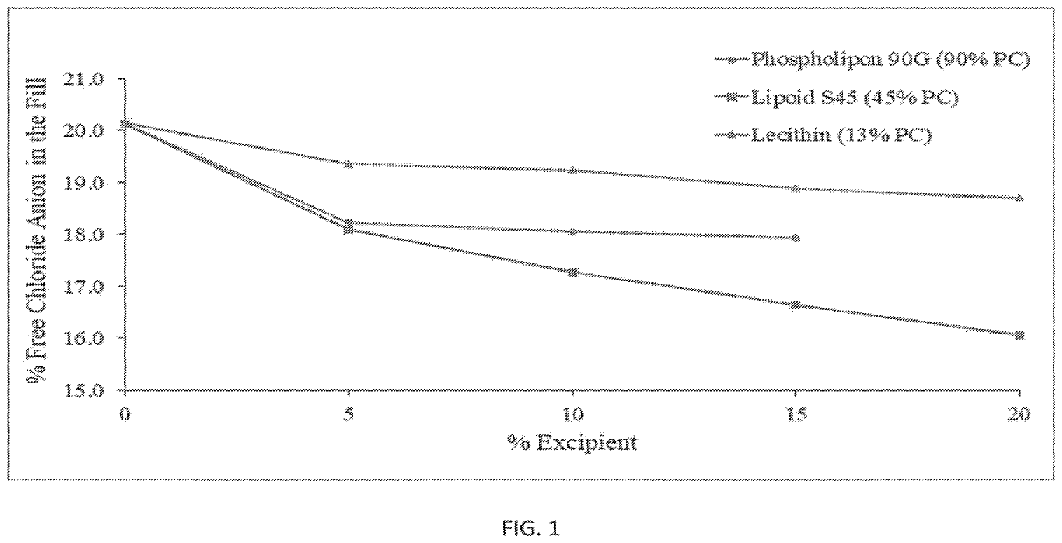

FIG. 1 depicts the free chloride anion chelating capabilities of different levels of PC in various lecithin grades. As shown in FIG. 1, using the liquid lecithin grade with approximately 13% PC, the free chloride ion was reduced to about 19% as compared to the initial 20%. As further shown in FIG. 1, using Lipoid S45, which contained at least 45% of PC, the free chloride ion was reduced to about 16% as compared to the initial 20%. FIG. 1 further demonstrated that using Phospholipon 90G, which contained 90% PC, the free chloride ion was reduced to about 18% as compared to the initial 20%. As shown in FIG. 1, lecithin (13% PC) and Lipoid S45 (45% PC) illustrated a continuous reduction in the % free chloride anion with increasing concentration of the corresponding excipient/chelating agent. In contrast. Phospholipon 90G (90% PC) appeared to reach a plateau after addition of 5% of the excipient. The samples evaluated in Example 1 had a molar ratio of PC to API of between 1:5 to 1:25.

### Example 2—Stability of Formulations with and without Lecithin

Fill material with and without lecithin was encapsulated into softgel capsules containing choline chloride. The capsules were subjected to an accelerated stability study conditions for an extended duration at a temperature of about 40° C.

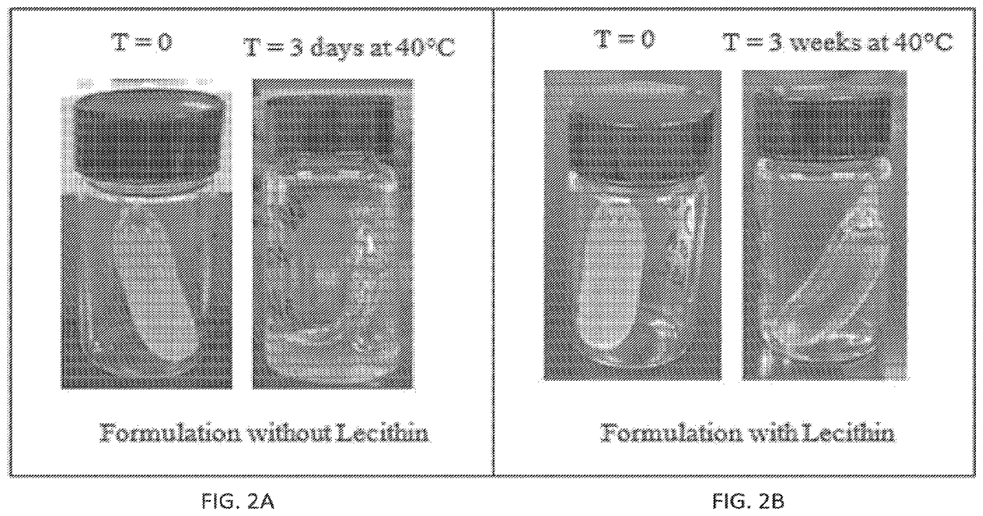

FIG. 2A depicts the softgel capsule formulations without lecithin before an accelerated stability study and after the accelerated stability study. FIG. 2B depicts a softgel capsule formulation with lecithin before an accelerated stability study and after the accelerated stability study.

Within three days, the test formulation without lecithin (FIG. 2A) dissolved the gelatin shell and resulted in leakage of the fill material at accelerated stability conditions (40° C.). Capsules containing formulation with lecithin retained the gelatin integrity at the accelerated stability study conditions (40° C.) after 3 weeks of storage. No fill material leakage was detected for formulation containing lecithin (FIG. 2B).

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”. “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A capsule comprising:
a fill material,
wherein the fill material comprises:
a salt form of a basic or acidic active pharmaceutical ingredient (API) and a free anion,
an anionic chelating agent comprising lecithin,
wherein the molar ratio of the anionic chelating agent to the free anion ranges from about 0.5 to about 3; and
a shell composition.

2. The capsule of claim 1, wherein the capsule maintains its integrity at 40° C., and 3 weeks of storage, wherein the integrity of the capsule is measured based on leakage of the fill material from the shell composition.

3. The capsule of claim 2, wherein the free anion is chloride, bromide, fluoride, sulfate, phosphate, formate, acetate, trifluoroacetate, maleate, tartrate, methanesulfonate, benzenesulfonate, nitrate, or a combination thereof.

4. The capsule of claim 1, wherein the fill material comprises from about 0.1 wt % to about 50 wt % API, based on total weight of the fill material.

5. The capsule of claim 1, wherein the fill material comprises from about 1 wt % to about 50 wt % anionic chelating agent, based on total weight of the fill material.

6. The capsule of claim 1, wherein the molar ratio of anionic chelating agent to the free anion range from about 0.8 to about 2.8.

7. The capsule of claim 1, wherein the lecithin comprises from about 10 wt % to about 95 wt % phospholipid components with positively charged functional groups, based on total weight of the lecithin.

8. The capsule of claim 1, wherein the lecithin comprises from about 10 wt % to about 95 wt % phosphatides comprising one or more of phosphatidylcholine (PC), phosphatidylethanolamine (PE), phosphatidylinositol (PI), and phosphatidylserine (PS).

9. (canceled)

10. (canceled)

11. The capsule of claim 1, wherein the fill material further comprises a lipid-based matrix comprising one or more of glycerides, triglycerides, semi-synthetic ester glycerides, fatty acids, alcohols, fatty acid esters, lipophilic surfactants, hydrophilic surfactants, carbohydrates, and co-solvents, and combinations thereof.

12. The capsule of claim 11, wherein the lipid-based matrix comprises vegetable oils.

13.-15. (canceled)

16. The capsule of claim 11, wherein the lipid-based matrix comprises fatty acids with the formulation R—C(═)—OH, wherein R is C₄-C₂₀, and wherein the fatty acids are fully saturated or contain one or more sites of unsaturation.

17. (canceled)

18. (canceled)

19. The capsule of claim 11, wherein the lipid-based matrix comprises one or more alcohols, fatty acid esters, and a combination thereof.

20. The capsule of claim 19, wherein the alcohols comprise one or more of ethanol, isopropanol, isobutanol, glycerol, propylene glycol.

21. (canceled)

22. The capsule of claim 19, wherein the fatty acid esters comprise mono-,di-, tri-esters of medium chain or long-chain fatty acid surfactants and/or co-solvents.

23. The capsule of claim 11, wherein the surfactants and/or co-solvents comprise polyoxyl 35 castor oil, polyoxyl 40 hydrogenated castor oil, polyethylene glycol, polysorbate 80, span 80, labrafil M2125, labrasol, gelucire 44/14, and combinations thereof.

24. The capsule of claim 1, wherein the capsule is a softgel capsule.

25.-27. (canceled)

28. A process of preparing a capsule of claim 1, the process comprising:
preparing the fill material; and
encapsulating the fill material with the shell composition.

29. A process of stabilizing a capsule comprising:
combining a salt form of a basic or acidic active pharmaceutical ingredient (API) and a free anion with an anionic chelating agent comprising lecithin at a molar ratio of anionic chelating agent to free anion of about 0.5 to about 3 to prepare a fill material; and
encapsulating the fill material in a shell composition.

30. The process of claim 29, wherein the capsule maintains its integrity at 40° C., and 3 weeks of storage, wherein the integrity of the capsule is measured based on leakage of the fill material from the shell composition.

31. A capsule comprising:
a fill material,
wherein the fill material comprises:
a salt form of a basic or acidic active pharmaceutical ingredient (API) and a free anion,
an anionic chelating agent comprising lecithin, wherein the lecithin comprises from about 10 wt % to about 95 wt % phospholipid components with positively charged functional groups, based on total weight of the lecithin; and
a shell composition,
wherein the capsule maintains its integrity at 40° C., and 3 weeks of storage, wherein the integrity of the capsule is measured based on leakage of the fill material from the shell composition.
